(19) United States
(12) Patent Application Publication
(10) Pub. No.: US 2017/0117931 A1
(43) Pub. Date: Apr. 27, 2017

(54) Wireless Communication for a Dialysis System

(71) Applicant: Fresenius Medical Care Holdings, Inc., Waltham, MA (US)

(72) Inventor: James Concepcion, Concord, CA (US)

(21) Appl. No.: 14/921,028

(22) Filed: Oct. 23, 2015

(51) Int. Cl. H04B 1/40; H04B 5/00; A61M 5/14; H04W 4/00; H01Q 1/22; A61M 1/14

(52) U.S. Cl. CPC H04B 1/40; H01Q 1/22; A61M 1/14; A61M 5/1415; H04W 4/008; H04B 5/0031; A61M 2205/3584; A61M 2205/3592; A61M 2205/3561; H04W 84/12

(57) ABSTRACT

A dialysis system comprising: a dialysis machine; a communication module for allowing the dialysis system to communicate using a wireless protocol; an antenna coupled to the communication module, the antenna for transmitting and receiving wireless signals according to the wireless protocol; and an intravenous (IV) pole configured to: support one or more containers of solution; and house at least a portion of the antenna.

### TECHNICAL FIELD

This invention relates to wireless communication for dialysis systems.

### BACKGROUND

Renal dysfunction or failure and, in particular, end-stage renal disease, causes the body to lose the ability to remove water and minerals and excrete harmful metabolites, maintain acid-base balance and control electrolyte and mineral concentrations within physiological ranges. Toxic uremic waste metabolites, including urea, creatinine, and uric acid, accumulate in the body's tissues which can result in a person's death if the filtration function of the kidney is not replaced.

Dialysis is commonly used to replace kidney function by removing these waste toxins and excess water. In one type of dialysis treatment—hemodialysis—toxins are filtered from a patient's blood externally in a hemodialysis machine. Blood passes from the patient through a dialyzer separated by a semi-permeable membrane from a large volume of externally-supplied dialysis solution. The waste and toxins dialyze out of the blood through the semi-permeable membrane into the dialysis solution, which is then typically discarded.

The dialysis solutions or dialysates used during hemodialysis typically contain sodium chloride and other electrolytes, such as calcium chloride or potassium chloride, a buffer substance, such as bicarbonate or acetate, and acid to establish a physiological pH, plus optionally, glucose or another osmotic agent.

Dialysis systems can be configured to access networks for receiving and providing information relevant to a dialysis treatment.

### SUMMARY

In one aspect, a dialysis system includes a dialysis machine and a communication module for allowing the dialysis system to communicate using a wireless protocol. The dialysis system also includes an antenna coupled to the communication module. The antenna is for transmitting and receiving wireless signals according to the wireless protocol. The dialysis system also includes an intravenous (IV) pole configured to support one or more containers of fluid. The IV pole is also configured to house at least a portion of the antenna.

Implementations can include one or more of the following features.

In some implementations, the IV pole includes an electrically conductive material.

In some implementations, the antenna includes at least a portion of the IV pole.

In some implementations, the IV pole is coupled to the antenna and is configured to enhance the transmitting and receiving ability of the antenna.

In some implementations, the IV pole is configured to increase a power gain of the antenna.

In some implementations, the IV pole is configured to increase signal strengths of wireless signals transmitted and received by the antenna.

In some implementations, the communication module is a wireless local area network (WLAN) card.

In some implementations, the communication module is a near field communication (NFC) initiator.

In some implementations, the communication module is a mobile broadband modem.

In some implementations, the communication module is a Bluetooth™ transceiver.

In another aspect, an IV pole for a dialysis machine is configured to support one or more containers of fluid. The IV pole is also configured to house at least a portion of an antenna for transmitting and receiving wireless signals according to a wireless protocol. The antenna is configured to be coupled to a communication module that allows the dialysis machine to communicate using the wireless protocol.

In another aspect, a dialysis system includes a dialysis machine and a communication module for allowing the dialysis system to communicate using a wireless protocol. The dialysis system also includes an intravenous (IV) pole coupled to the communication module. The IV pole is configured to support one or more containers of fluid. The IV pole is also configured to transmit and receive wireless signals according to the wireless protocol.

Implementations can include one or more of the following features.

In some implementations, the IV pole includes an antenna disposed within a channel formed by a body of the IV pole. The antenna is configured to transmit and receive wireless signals according to the wireless protocol.

Implementations can include one or more of the following advantages.

In some implementations, the positioning of the antenna near the top of the IV pole can enhance the transmitting and receiving ability of the antenna. For example, positioning the antenna in an elevated location can reduce the occurrences of physical objects blocking and/or interfering with the wireless signals

In some implementations, housing the antenna in the electrically conductive IV pole can increase the power gain of the antenna and the signal strengths of wireless signals transmitted and received by the antenna, thereby improving the range and reliability of wireless communications between the dialysis system and other devices.

The details of one or more implementations are set forth in the accompanying drawings and the description below. Other aspects, features, and advantages will be apparent from the description and drawings, and from the claims.

### DESCRIPTION OF DRAWINGS

FIG. 1 is a front perspective view of a hemodialysis system that includes a communication module and an antenna housed in an IV pole.

FIG. 2 shows another example of the antenna and IV pole of FIG. 1 in which a portion of the antenna is positioned outside of the IV pole.

FIG. 3 shows another example of an antenna and IV pole in which the antenna includes a portion of the IV pole.

FIG. 4 shows an example of a computer system for controlling the dialysis system.

### DETAILED DESCRIPTION

Medical devices can be configured to communicate with other devices using a wireless protocol. For example, a dialysis system can include a communication module, such as a wireless local area network (WLAN) card, for allowing the dialysis system to access a medical facility network and/or the internet. The communication module is typically located inside a housing of a dialysis machine. However, such positioning of the communication module may introduce various physical and/or electromagnetic impediments that can reduce the communication module's ability to effectively transmit and receive wireless signals, potentially resulting in weak and/or dropped wireless connections.

Dialysis systems typically include an intravenous (IV) pole for holding medical fluids for IV infusion into a dialysis patient. In some implementations, an antenna of the communication module can be positioned inside the IV pole to enhance the transmitting and receiving ability of the antenna. For example, the positioning of the antenna in an elevated location can improve the antenna's transmitting and receiving ability. In some implementations, the IV pole is made of an electrically conductive material (e.g., metal), and the antenna can include at least a portion of the IV pole. In this way, the IV pole may itself act as an antenna that is more powerful and/or efficient than a standard communication module antenna (e.g., a standard WLAN card antenna).

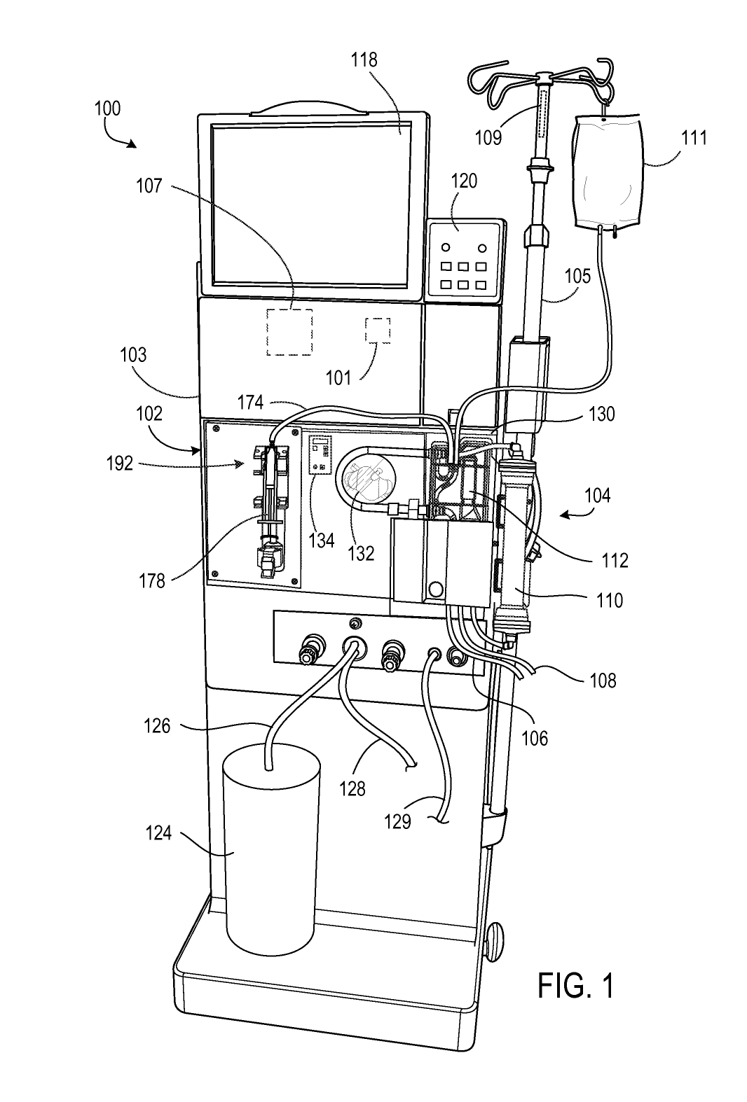

FIG. 1 shows a hemodialysis system 100 that is configured to communicate using a wireless protocol. The hemodialysis system 100 includes a hemodialysis machine 102 and an IV pole 105 that is configured to support one or more containers 111. The containers 111 can hold fluids (e.g., saline, medication, blood, dialysate, etc.) for IV infusion into a patient (not shown) or for infusion into the hemodialysis machine 102.

A disposable blood component set 104 that partially forms a blood circuit is connected to the hemodialysis machine 102. During hemodialysis treatment, an operator connects arterial and venous patient lines 106, 108 of the blood component set 104 to the patient. The blood component set 104 includes, among other things, a dialyzer 110 and an air release device 112. During the treatment, blood is circulated through the dialyzer 110 to be filtered. The air release device 112 vents air in the blood to the atmosphere to prevent air from being delivered into the patient's body.

The blood component set 104 is secured to a module 130 attached to the front of the hemodialysis machine 102. The module 130 includes the blood pump 132 capable of circulating blood through the blood circuit. The module 130 also includes various other instruments capable of monitoring the blood flowing through the blood circuit. The module 130 includes a door that when closed, as shown in FIG. 1, cooperates with the front face of the module 130 to form a compartment sized and shaped to receive the blood component set 104. In the closed position, the door presses certain blood components of the blood component set 104 against corresponding instruments exposed on the front face of the module 130.

The operator uses a blood pump control module 134 to operate the blood pump 132. The blood pump control module 134 includes a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in an arterial drip chamber.

The hemodialysis machine 102 further includes a dialysate circuit formed by the dialyzer 110, various other dialysate components, and dialysate lines connected to the hemodialysis machine 102. Many of these dialysate components and dialysate lines are inside the housing 103 of the hemodialysis machine 102 and are thus not visible in FIG. 1. During treatment, while the blood pump 132 circulates blood through the blood circuit, dialysate pumps (not shown) circulate dialysate through the dialysate circuit.

A dialysate container 124 is connected to the hemodialysis machine 102 via a dialysate supply line 126. A drain line 128 and an ultrafiltration line 129 also extend from the hemodialysis machine 102. The dialysate supply line 126, the drain line 128, and the ultrafiltration line 129 are fluidly connected to the various dialysate components and dialysate lines inside the housing 103 of the hemodialysis machine 102 that form part of the dialysate circuit. During hemodialysis, the dialysate supply line 126 carries fresh dialysate from the dialysate container 124 to the portion of the dialysate circuit located inside the hemodialysis machine 102. As noted above, the fresh dialysate is circulated through various dialysate lines and dialysate components, including the dialyzer 110, that form the dialysate circuit. As will be described below, as the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate (described below) and excess fluid drawn from the patient is carried to the drain via the ultrafiltration line 129.

The dialyzer 110 serves as a filter for the patient's blood. The dialysate passes through the dialyzer 110 along with the blood, as described above. A semi-permeable structure (e.g., a semi-permeable membrane and/or semi-permeable microtubes) within the dialyzer 110 separates blood and dialysate passing through the dialyzer 110. This arrangement allows the dialysate to collect toxins from the patient's blood. The filtered blood exiting the dialyzer 110 is returned to the patient. The dialysate exiting the dialyzer 110 includes toxins removed from the blood and is commonly referred to as “spent dialysate.” The spent dialysate is routed from the dialyzer 110 to a drain.

A drug pump 192 also extends from the front of the hemodialysis machine 102. The drug pump 192 is a syringe pump that includes a clamping mechanism configured to retain a syringe 178 of the blood component set 104. The drug pump 192 also includes a stepper motor configured to move the plunger of the syringe 178 along the axis of the syringe 178. A shaft of the stepper motor is secured to the plunger in a manner such that when the stepper motor is operated in a first direction, the shaft forces the plunger into the syringe, and when operated in a second direction, the shaft pulls the plunger out of the syringe 178. The drug pump 192 can thus be used to inject a liquid drug (e.g., heparin) from the syringe 178 into the blood circuit via a drug delivery line 174 during use, or to draw liquid from the blood circuit into the syringe 178 via the drug delivery line 174 during use.

The hemodialysis machine 102 includes a user interface with input devices such as a touch screen 118 and a control panel 120. The touch screen 118 and the control panel 120 allow the operator to input various different treatment parameters to the hemodialysis machine 102 and to otherwise control the hemodialysis machine 102. The touch screen 118 displays information to the operator of the hemodialysis system 100.

The hemodialysis machine 102 also includes a control unit 101 (e.g., a processor) configured to receive signals from and transmit signals to the touch screen 118, the control panel 120, and a communication module 107 (e.g., a WLAN card). The control unit 101 can control the operating parameters of the hemodialysis machine 102, for example, based at least in part on the signals received by the touch screen 118, the control panel 120, and the communication module 107.

The communication module 107 is configured to allow the hemodialysis system 100 to communicate using a wireless protocol. For example, the communication module 107 allows the hemodialysis machine 102 to wirelessly access a network (e.g., a medical facility network, the internet, etc.).

An antenna 109 is housed in the IV pole 105 and is coupled to the communication module 107 to facilitate and enhance wireless communication. The antenna 109 may reside within a channel formed by a body of the IV pole 105. The antenna 109 may be coupled to the communication module 107 by wires. The antenna 109 may be made from a conductive material such as copper, aluminum, or silver, among others. The antenna 109 is configured to transmit and receive wireless signals according to the wireless protocol. The positioning of the antenna 109 in an elevated location (e.g., near the top of the IV pole 105) can enhance the transmitting and receiving ability of the antenna 109, for example, by reducing the occurrences of physical objects blocking and interfering with the wireless signals. In this way, the antenna 109 can receive relatively weak wireless signals that might otherwise not be capable of being received (e.g., if the antenna 109 were internal to the hemodialysis machine 102). Similarly, the antenna 109 can transmit wireless signals that do not need to pass through the housing 103 of the hemodialysis machine 102.

The transmitting and receiving ability of the antenna 109 may be based on the position of the antenna 109. For example, an antenna positioned ten feet from the ground may be capable of receiving more wireless signals or higher quality wireless signals than an antenna positioned two feet from the ground. The IV pole 105 can have a height that is sufficient for allowing the antenna 109 to be appropriately positioned. In some implementations, the IV pole 105 has a height of 4-10 feet (e.g., 8 feet). The antenna 109 can be positioned towards the top of the IV pole 105. For example, the antenna 109 can be positioned in or along the uppermost ½, ⅓, or ¼ of the IV pole 105.

For some applications, it may be especially beneficial for the hemodialysis system 100 to reliably receive and transmit information wirelessly. For example, the communication module 107 may allow the hemodialysis system 100 to access patient information that is stored on a medical facility database. Patient information can include the patient's name, identification number, address, phone number, medical history, treatment history, treatment prescriptions, treatment parameters to be used for particular treatments (e.g., dialysate type, dialysate fill volume, dialysate flow rate, etc.), and the like. The hemodialysis system 100 can use the received information to identify a particular treatment that corresponds to the particular patient and cause the hemodialysis machine 102 to carry out that treatment. For example, the hemodialysis system 100 can identify treatment parameters included in the dialysis treatment and identify particular values to be used for those treatment parameters. The control unit 101 can cause the hemodialysis machine 102 to carry out the dialysis treatment based on the identified treatment parameters. A reliable wireless communication system ensures that such patient information is considered in formulating the treatment.

The wireless systems described herein can be used to carry out individual treatments in an efficient manner. For example, suppose a patient has a medical condition that requires an atypical dialysis treatment. Perhaps the patient's treatment requires an abnormally high dialysate flow rate. The hemodialysis system 100 wirelessly accesses a medical database using the communication module 107 to receive patient information. The patient information includes the patient's medical history, treatment prescriptions, and treatment parameters. In particular, the treatment prescription includes instructions for causing the hemodialysis machine 102 to employ the abnormally high dialysate flow rate that the patient requires. Such information is provided to the control unit 101, and the control unit 101 causes the appropriate treatment to be administered. For example, the control unit 101 can cause the hemodialysis machine 102 to operate a pump (e.g., a dialysate pump) such that the required dialysate flow rate is achieved.

The communication module 107 can also allow the hemodialysis system 100 to provide information to the medical facility database, for example, following a dialysis treatment. The information can include data related to the patient's dialysis treatment, such as treatment results and/or treatment details (e.g., treatment runtime, drugs administered, particular treatments/functions performed, etc.).

While certain implementations have been described, other implementations are possible.

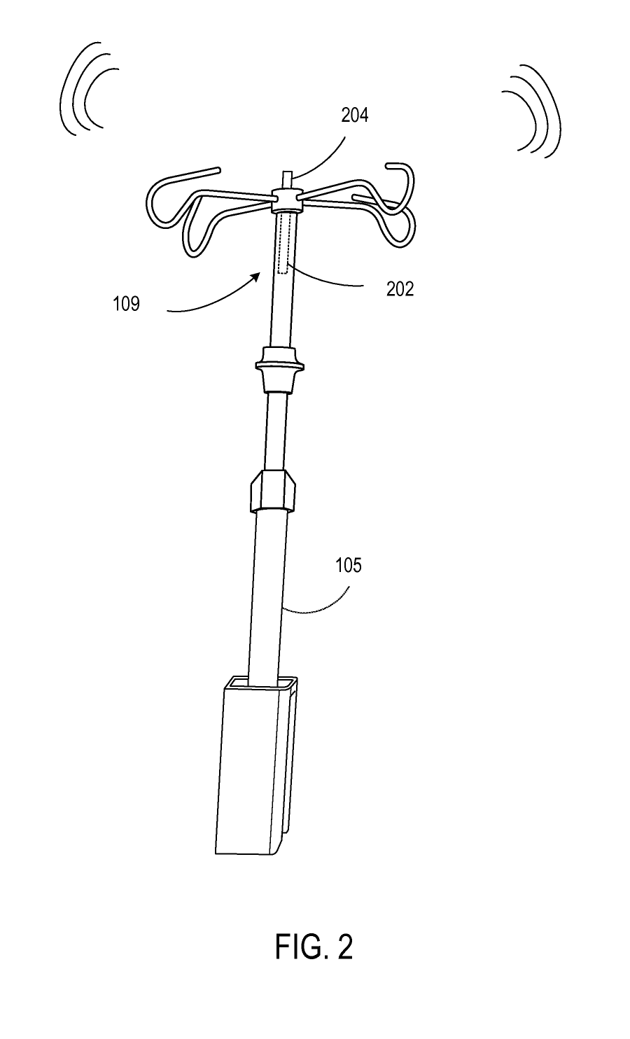

Referring to FIG. 2, in some implementations, a first portion 202 of the antenna 109 is housed in the IV pole 105 and a second portion 204 of the antenna 109 is positioned outside of the IV pole 105. Such a configuration can enhance the transmitting and receiving ability of the antenna by creating a clear path for the second portion 204 to transmit and receive wireless signals. While the second portion 204 of the antenna 109 is situated above the first portion 202 of the antenna 109 in the illustrated example, other configurations are possible. For example, in some implementations, the second portion 204 of the antenna 109 may extend from a side surface of the IV pole 105. In some implementations, the second portion 204 of the antenna 109 is aimed towards an expected source of wireless signals (e.g., a wireless router).

In some implementations, the IV pole is made of an electrically conductive material (e.g., metal), and the antenna includes at least a portion of the IV pole. In this way, the IV pole may itself act as an antenna that is more powerful and/or efficient than a standard communication module antenna.

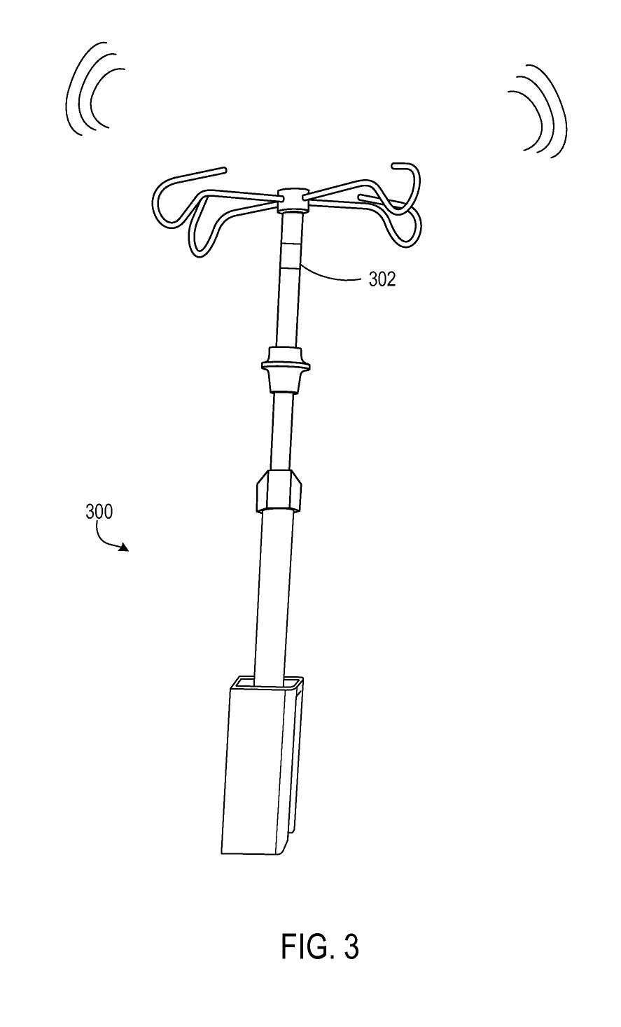

FIG. 3 shows an example of an IV pole 300 that is made of an electrically conductive material such as copper, aluminum, or silver, among others. A portion of the IV pole 300 acts as an antenna 302. In other words, the antenna 302 includes a portion of the IV pole 300. The antenna portion 302 can be coupled to the rest of the IV pole 300 by wires (not shown), or the antenna portion 302 can be a contiguous portion of the conductive IV pole 300.

In some implementations, the IV pole can be directly coupled to the communication module such that the IV pole itself is the antenna. Such a configuration provides for an antenna that contains a relatively large amount of conductive material. The additional conductive material can allow the antenna to transmit higher-strength signals and receive signals that may otherwise be too weak to be detected. The additional conductive material can also cause the antenna to have increased efficiency, thereby resulting in improved (e.g., increased) power gain.

In some implementations, the antenna is specially designed or tuned for transmitting and receiving particular types of wireless signals (e.g., wireless signals according to a particular protocol). For example, the antenna may have dimensions that are based on the wavelength of the wireless signals to be received and transmitted by the antenna. In some implementations, the antenna has a length that is chosen to improve the resonance of the antenna, thereby improving the antenna's efficiency. For example, the antenna may have a length of ¼ or ½ of the wavelength of expected wireless signals. In some implementations, the antenna has a length of 1-3 inches.

While the communication module of the hemodialysis machine has been shown as being positioned at a particular location within the housing of the dialysis machine, the communication module may be positioned elsewhere in the hemodialysis system. For example, the communication module may be positioned at or near the IV pole. Such positioning may reduce the length of wires that couple the antenna to the communication module. The use or shorter wires may reduce the chance of the wires negatively impacting the operating characteristics of the antenna.

While the communication module has largely been described as being a WLAN card and the antenna has largely been described as operating according to a WLAN protocol, other wireless protocols can also be used. In some implementations, the communication module is a near field communication (NFC) initiator that is configured to allow the dialysis system to communicate using an NFC protocol. In some implementations, the communication module is a broadband modem or a Bluetooth™ transceiver that is configured to allow the dialysis system to communicate using a broadband or Bluetooth™ protocol, respectively.

While the communication module has been largely described as allowing the dialysis system to communicate with a network, in some implementations, the communication module configured to communicate with other medical devices (e.g., dialysis machines, dialysis machine components, dialysis machine accessories, etc.). In some implementations, the communication module allows the dialysis system to communicate with computer systems, servers, and/or databases associated with a particular medical facility.

While the dialysis system has been largely described as being a hemodialysis system, other medical treatment systems can employ the techniques described herein. Examples of other medical treatment systems include peritoneal (PD) dialysis systems, hemofiltration systems, hemodiafiltration systems, apheresis systems, cardiopulmonary bypass systems, and drug infusion systems.

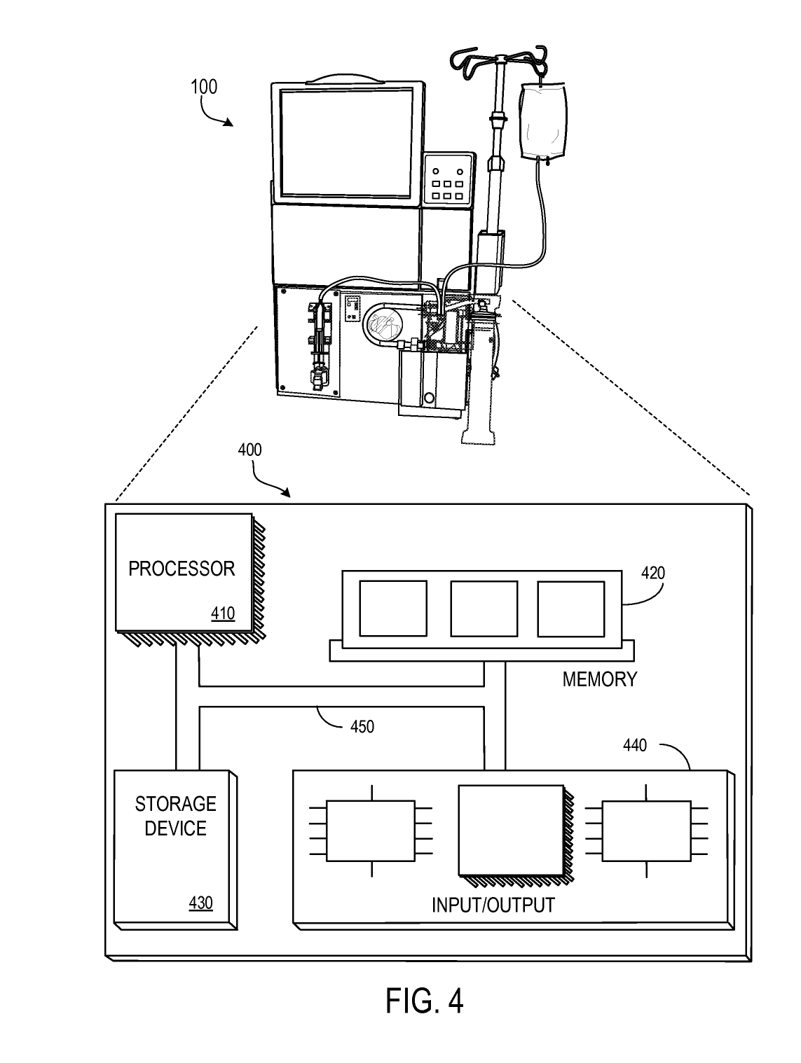

FIG. 4 is a block diagram of an example computer system 400. For example, the control unit 101 of the hemodialysis machine 102 (shown in FIG. 1) could be an example of the system 400 described here. The system 400 includes a processor 410, a memory 420, a storage device 430, and an input/output device 440. Each of the components 410, 420, 430, and 440 can be interconnected, for example, using a system bus 450. The processor 410 is capable of processing instructions for execution within the system 400. The processor 410 can be a single-threaded processor, a multi-threaded processor, or a quantum computer. The processor 410 is capable of processing instructions stored in the memory 420 or on the storage device 430. The processor 410 may execute operations such as causing the dialysis system to carry out functions related to voice commands, voice alarms, and voice instructions.

The memory 420 stores information within the system 400. In some implementations, the memory 420 is a computer-readable medium. The memory 420 can, for example, be a volatile memory unit or a non-volatile memory unit. In some implementations, the memory 420 stores information (e.g., text) that corresponds to one or more voice commands and/or wakeup commands, profiles that define arrangements of buttons to be displayed by a user interface (e.g., the touch screen display 118), authentication information that identifies access privileges of various users of the dialysis system 100, and/or information related to verbosity settings.

The storage device 430 is capable of providing mass storage for the system 400. In some implementations, the storage device 430 is a non-transitory computer-readable medium. The storage device 430 can include, for example, a hard disk device, an optical disk device, a solid-date drive, a flash drive, magnetic tape, or some other large capacity storage device. The storage device 430 may alternatively be a cloud storage device, e.g., a logical storage device including multiple physical storage devices distributed on a network and accessed using a network.

The input/output device 440 provides input/output operations for the system 400. In some implementations, the input/output device 440 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). In some implementations, the input/output device includes driver devices configured to receive input data and send output data to other input/output devices, e.g., keyboard, printer and display devices (such as the touch screen display 118). In some implementations, mobile computing devices, mobile communication devices, and other devices are used.

In some implementations, the system 400 is a microcontroller. A microcontroller is a device that contains multiple elements of a computer system in a single electronics package. For example, the single electronics package could contain the processor 410, the memory 420, the storage device 430, and input/output devices 440.

Although an example processing system has been described in FIG. 4, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

A number of embodiments of the invention have been described. Nevertheless, it will be understood that various modifications may be made without departing from the spirit and scope of the invention. Accordingly, other embodiments are within the scope of the following claims.

1. A dialysis system comprising:
a dialysis machine;
a communication module for allowing the dialysis system to communicate using a wireless protocol;
an antenna coupled to the communication module, the antenna adapted to transmit and receive wireless signals according to the wireless protocol; and
an intravenous (IV) pole configured to:
support one or more containers of fluid; and
house at least a portion of the antenna,
wherein the antenna has a length that is proportional to a wavelength of the wireless signals transmitted and received according to the wireless protocol, the length chosen to optimize the antenna for transmitting and receiving the wireless signals according to the wireless protocol.

2. The dialysis system of claim 1, wherein the IV pole comprises an electrically conductive material.

3. The dialysis system of claim 2, wherein the antenna comprises at least a portion of the IV pole.

4. The dialysis system of claim 2, wherein the IV pole is coupled to the antenna and is configured to enhance the transmitting and receiving ability of the antenna.

5. The dialysis system of claim 4, wherein the IV pole is configured to increase a power gain of the antenna.

6. The dialysis system of claim 4, wherein the IV pole is configured to increase signal strengths of wireless signals transmitted and received by the antenna.

7. The dialysis system of claim 1, wherein the communication module is a wireless local area network (WLAN) card.

8. The dialysis system of claim 1, wherein the communication module is a near field communication (NFC) initiator.

9. The dialysis system of claim 1, wherein the communication module is a mobile broadband modem.

10. The dialysis system of claim 1, wherein the communication module is a Bluetooth™ transceiver.

11. An IV pole for a dialysis machine, the IV pole configured to:
support one or more containers of fluid; and
house at least a portion of an antenna adapted to transmit and receive wireless signals according to a wireless protocol,
wherein the antenna has a length that is proportional to a wavelength of the wireless signals transmitted and received according to the wireless protocol, the length chosen to optimize the antenna for transmitting and receiving the wireless signals according to the wireless protocol, and the antenna is configured to be coupled to a communication module that allows the dialysis machine to communicate using the wireless protocol.

12. A dialysis system comprising:
a dialysis machine;
a communication module for allowing the dialysis system to communicate using a wireless protocol; and
an intravenous (IV) pole comprising an antenna, the IV pole coupled to the communication module and configured to:
support one or more containers of fluid; and
transmit and receive wireless signals according to the wireless protocol,
wherein the antenna has a length that is proportional to a wavelength of the wireless signals transmitted and received according to the wireless protocol, the length chosen to optimize the antenna for transmitting and receiving the wireless signals according to the wireless protocol.

13. The dialysis system of claim 12, wherein the antenna is disposed within a channel formed by a body of the IV pole.

14. The dialysis system of claim 1, wherein a first portion of the antenna is housed in the IV pole, and a second portion of the antenna is positioned outside of the IV pole and extends beyond a top end of the IV pole.
